SEQUENCE LISTING (11 sequences)

SEQ ID NO: 1 - protein, 5 aa, synthetic construct
  source 1..5: note = HCDR1
DYYMY

SEQ ID NO: 2 - protein, 17 aa, synthetic construct
  source 1..17: note = HCDR2
YITKGGGSTYYPDSVKG

SEQ ID NO: 3 - protein, 10 aa, synthetic construct
  source 1..10: note = HCDR3
QTNYDFTMDY

SEQ ID NO: 4 - protein, 11 aa, synthetic construct
  source 1..11: note = LCDR1
KASQDVGTSVA

SEQ ID NO: 5 - protein, 7 aa, synthetic construct
  source 1..7: note = LCDR2
WASARHT

SEQ ID NO: 6 - protein, 9 aa, synthetic construct
  source 1..9: note = LCDR3
QQYSSYPLT

SEQ ID NO: 7 - protein, 119 aa, synthetic construct
  source 1..119: note = VH
EVQLVESGGGLVQPGGSLRLSCAASGFTFSDYYMYWVRQAPGKGLEWVAYITKGGGSTYYPDSVKGRFTISRDNAKNTLYLQMNSLRAEDTAVYYCARQTNYDFTMDYWGQGTLVTVSS

SEQ ID NO: 8 - protein, 107 aa, synthetic construct
  source 1..107: note = VL
EIVMTQSPATLSVSPGERATLSCKASQDVGTSVAWYQQKPGQAPRLLIYWASARHTGIPARFSGSGSGTEFTLTISSLQSEDFAVYYCQQYSSYPLTFGGGTKVEIK

SEQ ID NO: 9 - protein, 449 aa, synthetic construct
  source 1..449: note = Heavy chain
EVQLVESGGGLVQPGGSLRLSCAASGFTFSDYYMYWVRQAPGKGLEWVAYITKGGGSTYYPDSVKGRFTISRDNAKNTLYLQMNSLRAEDTAVYYCARQTNYDFTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
  source 1..214: note = light chain
EIVMTQSPATLSVSPGERATLSCKASQDVGTSVAWYQQKPGQAPRLLIYWASARHTGIPARFSGSGSGTEFTLTISSLQSEDFAVYYCQQYSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 330 aa, synthetic construct
  source 1..330: note = IgG1 WT
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0304682 A1
(43) Pub. Date: Oct. 2, 2025

(54) FORMULATIONS CONTAINING ANTI-TIGIT ANTIBODY AND METHODS OF USE THEREOF

(71) Applicant: BeiGene Switzerland GmbH, Basel (CH)

(72) Inventors: Xiaoqing Jin, Suzhou (CN); Sufang GU, Suzhou (CN); Yu JI, Suzhou (CN); Jian SHEN, Suzhou (CN); Kai XU, Suzhou (CN); Jinmei LI, Suzhou (CN); Jun WU, Suzhou (CN); Yuanyuan ZHANG, Suzhou (CN); Aijun DI, Suzhou (CN); Bo QIU, Guangzhou (CN)

(73) Assignee: BeiGene Switzerland GmbH, Basel (CH)

(21) Appl. No.: 19/176,565

(22) Filed: Apr. 11, 2025

Related U.S. Application Data
(63) Continuation of application No. PCT/CN2023/124732, filed on Oct. 16, 2023.

(30) Foreign Application Priority Data
Oct. 17, 2022 (WO) PCT/CN2022/125633

(51) Int. Cl. C07K 16/28; A61K 9/10; A61K 47/10; A61K 47/22; A61K 47/26

(52) U.S. Cl. CPC C07K 16/2803; A61K 9/10; A61K 47/10; A61K 47/22; A61K 47/26; C07K 2317/24

(57) ABSTRACT

The present invention relates generally to the field of pharmaceutical formulations of antibodies against T cell immunoreceptor with Ig and ITIM domains (TIGIT), or antigen binding fragments thereof. The pharmaceutical formulations of the present invention exhibit a substantial degree of anti-TIGIT antibody stability after being subjected to stress conditions, accelerated and long-term storage. Also provided are methods of making and methods of using such antibody formulations.

### CROSS-REFERENCE TO RELATED APPLICATIONS

This application is a continuation of International Patent Application No. PCT/CN2023/124732, filed Oct. 16, 2023, which claims priority from International Patent Application No. PCT/CN2022/125633, filed Oct. 17, 2022. The contents of these applications are incorporated herein by reference in their entireties.

### SEQUENCE LISTING

The instant application contains a sequence listing which has been submitted electronically in xml format, and is hereby incorporated by reference in its entirety. Said xml file was created on Jan. 23, 2024, is 14 bytes in size, and named 138881_1299_Sequence_Listing.xml.

### FIELD OF THE DISCLOSURE

Disclosed herein are stable formulations comprising antibodies or antigen binding fragments thereof that binds to T cell immunoreceptor with Ig and ITIM domains (TIGIT). Also disclosed herein are methods of preparing the formulations and treating cancers with the formulations of the present disclosure.

### BACKGROUND OF THE DISCLOSURE

TIGIT, is a newly discovered co-stimulatory molecule with immunosuppressive effect in recent years. TIGIT is mainly expressed in T cells and NK cells, and directly inhibits the killing effect of T cells and NK cells on tumor cells through the ITIM region. Similar to the inhibitory receptors CTLA4 and PD-1, TIGIT also plays an important role in autoimmunity, so TIGIT has become another highly potential immunotherapy target.

Monoclonal antibody drugs are injected intravenously or subcutaneously. Antibodies can degrade and/or aggregate due to physical and chemical instability during storage, transportation, and use, resulting in a reduction of biological activity and an increase in immunogenicity. Physical instability includes denaturation, aggregation, and/or precipitation of antibodies. Chemical instability comes from deamidation, isomerization, oxidation, or hydrolysis. In order to improve these aspects, some antibody formulations have added additional stabilizers, antioxidants, preservatives, etc. to improve the stability of antibodies, but the introduction of too many excipients increases the chance of infusion reactions to the patient. Therefore, the appropriate formulation of the antibody can keep the antibody physically and chemically stable.

### SUMMARY OF THE INVENTION

The invention provides for an anti-TIGIT antibody pharmaceutical formulation.

A pharmaceutical formulation comprising:
about 5 mg/mL to about 200 mg/mL of an anti-TIGIT antibody, or antigen binding fragment thereof;
about 5 mM to about 50 mM formulation buffer providing a pH of about 5.0 to about 7.0;
about 30 mM to about 300 mM stabilizer;
about 0.01 mg/ml to about 1 mg/ml non-ionic surfactant.

The formulation, wherein the anti-TIGIT antibody or antigen binding fragment thereof, comprises a heavy chain variable region that comprises a HCDR1 (Heavy Chain Complementarity Determining Region 1) of SEQ ID NO: 1, a HCDR2 of SEQ ID NO: 2 and a HCDR3 of SEQ ID NO: 3 and a light chain variable region that comprises: a LCDR1 (Light Chain Complementarity Determining Region 1) of SEQ ID NO: 4, a LCDR2 of SEQ ID NO: 5 and a LCDR3 of SEQ ID NO: 6 . . .

The formulation, wherein the anti-TIGIT antibody or antigen binding fragment thereof, comprises SEQ ID NO:7 and SEQ ID NO:8.

The formulation, wherein the formulation buffer is selected from the group consisting of histidine, acetate, citrate, succinate, phosphate, mixture of histidine and acetic acid, or mixture of histidine and citric acid.

The formulation, wherein the formulation buffer is histidine.

The formulation, wherein the concentration of histidine buffer is 10 mM to 30 mM.

The formulation, wherein the formulation comprises 20 mM histidine buffer.

The formulation, wherein the pH is a range of pH 5.2-6.2.

The formulation, wherein the stabilizer is selected from the group consisting of trehalose, sucrose, sorbitol, mannitol, maltose, dextran, (2-hydroxypropyl)-b-cyclodextrin, sodium chloride, magnesium chloride, calcium chloride, sodium sulfate, sodium dihydrogen phosphate, or disodium hydrogen phosphate.

The formulation, wherein the stabilizer is trehalose.

The formulation, wherein the trehalose concentration is from 50 mM to 280 mM.

The formulation, wherein the trehalose concentration is from 150 mM to 250 mM.

The formulation, wherein the stabilizer is sucrose.

The formulation, wherein the sucrose concentration is from 50 mM to 280 mM.

The formulation, wherein the sucrose concentration is from 150 mM to 250 mM.

The formulation, wherein the non-ionic surfactant is selected from the group consisting of polysorbate 20, polysorbate 80 or poloxamer188.

The formulation, wherein the concentration of polysorbate 20 is from 0.1 mg/ml to 0.8 mg/ml.

The formulation, wherein polysorbate 20 concentration is from 0.2 mg/ml to 0.6 mg/ml.

The formulation, wherein the concentration of polysorbate 80 is from 0.1 mg/ml to 0.8 mg/ml.

The formulation, wherein polysorbate 80 concentration is from 0.2 mg/ml to 0.6 mg/ml.

The formulation, wherein the concentration of poloxamer 188 is from 0.1 mg/ml to 0.8 mg/ml.

The formulation, wherein poloxamer 188 concentration is from 0.2 mg/ml to 0.6 mg/ml.

The formulation, wherein the formulation comprises 30 mM acetic acid-sodium acetate, 240 mM sucrose, and 0.2 mg/ml polysorbate 80 with a pH of pH 5.5.

The formulation, wherein the formulation comprises 20 mM Histidine-Histidine HCl, 240 mM trehalose and 0.2 mg/ml polysorbate 20, with a pH of pH 5.8.

The formulation, wherein the formulation comprises 20 mM Histidine-Histidine HCl, 70 mM NaCl, 80 mM trehalose and 0.8 mg/ml polysorbate 20, with a pH of pH 6.0.

The formulation, wherein the concentration of the anti-TIGIT antibody, or antigen binding fragment thereof is from about 10 mg/mL to 150 mg/mL.

A method of making an antibody formulation, the method comprising:
a. exchanging the anti-TIGIT antibody to about 5 mM to about 50 mM buffer providing a pH of about 5.0 to about 7.0;
b. concentrating the antibody formulation of (a) to an antibody concentration of about 5-200 mg/mL;
c. adding non-ionic surfactant to the antibody formulation of (c) to achieve an antibody formulation having a concentration of surfactant of no less than 0.01 mg/ml.
d. adding stabilizer to the antibody to achieve an antibody formulation having a concentration of stabilizer no less than 30 mM, wherein the anti-TIGIT antibody comprises a heavy chain variable region that comprises a HCDR1 (Heavy Chain Complementarity Determining Region 1) of SEQ ID NO: 1, a HCDR2 of SEQ ID NO: 2 and a HCDR3 of SEQ ID NO: 3 and a light chain variable region that comprises: a LCDR1 (Light Chain Complementarity Determining Region 1) of SEQ ID NO: 4, a LCDR2 of SEQ ID NO: 5 and a LCDR3 of SEQ ID NO: 6.

The formulation, wherein the anti-TIGIT antibody or antigen binding fragment thereof, comprises SEQ ID NO:7 and SEQ ID NO:8.

A method for treating cancer in a human patient in need thereof comprising administering an effective amount of an anti-TIGIT antibody formulation of claim 1.

The method, wherein the anti-TIGIT antibody formulation is administered at a dose of about 200 mg to about 2400 mg.

The method, wherein the anti-TIGIT antibody formulation is administered once every three weeks.

The method, wherein the human patient is administered at least one other therapeutic agent selected from the group consisting of: zanubrutinib, pamiparib, tislelizumab, an anti-LAG3 antibody, a second anti-TIGIT antibody, an anti-4-1BB antibody, an anti-OX40 antibody, an anti-TIM-3 antibody, a CD40 agonist, a TLR agonist, a CAR-T cell, or a chemotherapeutic agent.

In some embodiments, the antibody formulation comprises an anti-TIGIT antibody, or antigen binding fragment thereof, a formulation buffer, a stabilizer, and a non-ionic surfactant. In some embodiments, the formulation buffer provides a pH range of between 5.0 and 7.0. In some embodiments, the antibody formulation is stable upon freeze-thaw and thermal stress.

In some embodiments, the antibody formulation can comprise or consist essentially of between about 10 mg/mL to about 40 mg/mL anti-TIGIT antibody or antigen binding fragment thereof, a formulation buffer, a stabilizer, and a non-ionic surfactant, and has a pH range between 5.2 and 6.2. In some embodiments, the antibody formulation is stable under stress conditions, accelerated and long-term storage.

In some embodiments, the formulation buffer is selected from the group consisting of histidine, acetate, citrate, succinate, phosphate, mixture of histidine and acetic acid, mixture of histidine and citric acid or any combination of them. In some embodiments, the formulation buffer can be histidine buffer. In some embodiments, the concentration of histidine buffer is from about 10 mM to about 30 mM. In some embodiments, the concentration of the histidine buffer is about 20 mM histidine.

In some embodiments, the stabilizer is selected from the group consisting of trehalose, sucrose, sorbitol, mannitol, maltose, dextran, (2-hydroxypropyl)-b-cyclodextrin, sodium chloride, magnesium chloride, calcium chloride, sodium sulfate, sodium dihydrogen phosphate, or disodium hydrogen phosphate. In some embodiments, the stabilizer can be trehalose. In some embodiments the trehalose is a, a-trehalose dihydrate. In other embodiments, the stabilizer can be sucrose. In some embodiments, the concentration of stabilizer can be from about 30 mM to about 300 mM. In some embodiments, the concentration of stabilizer can be from about 50 mM to about 280 mM, preferably about 150 mM, about 170 mM, about 190 mM, about 210 mM, about 230 mM, or about 250 mM.

In some embodiments, the non-ionic surfactant is selected from the group consisting of polysorbate 80 (PS80), polysorbate 20 (PS20) or poloxamer188 (P188). In some embodiments, the concentration of non-ionic surfactant can be from about 0.1 mg/ml to about 0.8 mg/ml. In some embodiments, the concentration of non-ionic surfactant is about 0.2 mg/ml, about 0.3 mg/ml, about 0.4 mg/ml, about 0.5 mg/ml, about 0.6 mg/ml. In some embodiments, the non-ionic surfactant is polysorbate 20. In some embodiments, the non-ionic surfactant is polysorbate 80. In some embodiments, the non-ionic surfactant is poloxamer 188.

In some embodiments, the antibody formulation consists essentially of about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL of an anti-TIGIT antibody, or antigen binding fragment thereof, about 20 mM histidine buffer, about 240 mM a, a-trehalose dihydrate or sucrose, about 0.2 mg/ml to about 0.6 mg/ml polysorbate 20 or polysorbate 80 or poloxamer 188, and the antibody formulation is of a pH 5.8±0.4.

Also provided herein are methods of making a stable anti-TIGIT antibody formulation, the method comprising: exchanging the anti-TIGIT antibody to about 5 mM to about 50 mM buffer providing a pH of about 5.0 to about 7.0; concentrating the antibody formulation from 5-200 mg/mL; adding a stabilizer to the antibody to achieve an antibody formulation having a concentration of stabilizer no less than 30 mM; adding a non-ionic surfactant to achieve an antibody formulation having a concentration of surfactant of no less than 0.001% (w/v).

Also provided herein are methods of reducing cancer growth in a human patient who has cancer, comprising administration to the patient an effective amount of the antibody formulation as described herein.

Provided herein are methods of reducing cancer growth in a human patient, comprising administration to the patient an effective amount of the antibody formulation as described herein.

In some embodiments, the antibody formulation has an antibody concentration between about 200 mg to 2400 mg. In another embodiment the antibody formulation has an antibody concentration of about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg or about 2400 mg. In some embodiments, the antibody formulation is administered once every three weeks. In some embodiments, the antibody formulation is about 400 mg to 1200 mg and is administered once every three weeks.

In some embodiments, the disclosure provides for methods of treating cancer with an anti-TIGIT subcutaneous antibody formulation in combination with another therapeutic agent. The other therapeutic agent is, for example, zanubrutinib, pamiparib, tislelizumab (BGB-A317), an anti-LAG3 antibody, a second anti-TIGIT antibody, an anti-4-1BB antibody, an anti-OX40 antibody, an anti-TIM-3 antibody, a CD40 agonist, a TLR agonist, a CAR-T cell, or a chemotherapeutic agent.

### BRIEF DESCRIPTION OF THE DRAWINGS

FIGS. 1A-C shows the results of different pH and buffers on stability from 5 cycles of freeze-thaw conditions (noted as “5 FT” in graphs). FIG. 1A is subvisible particles data, FIG. 1B is turbidity data and FIG. 1C is SEC monomer % data.

FIGS. 2A-C shows the results of different protein concentrations on stability from 5 cycles of freeze-thaw conditions (noted as “5 FT” in graphs). FIG. 2A is subvisible particles data, FIG. 2B is turbidity data and FIG. 2C is SEC monomer % data.

FIGS. 3A-E shows the results of different surfactants on stability in different stress conditions: 3 cycles of freeze-thaw (noted as “3 FT” in graphs), shaking for 2 days (noted as “Shake 2D” in graphs), photostability for 2 weeks (noted as “Photo 2W” in graphs) and thermal stress of the formulation kept at 37° C. for 4 weeks (noted as “37C4W” in graphs). FIG. 3A is subvisible particles data, FIG. 3B is turbidity data, FIG. 3C is SEC monomer % data, FIG. 3D is IEC main peak % data and FIG. 3E is CE-SDS (NR) intact peak % data.

FIGS. 4A-C shows the results of stability under stress conditions: thermal stress keeping the antibody formulation at 40° C. for 2 weeks (noted as “40C2W” in graphs), photostability of the formulation at 2 weeks (noted as “Photo 2W” in graphs), freeze-thaw (noted as “6 FT” in graphs). FIG. 4A is subvisible particles data, FIG. 4B is SEC monomer % data and FIG. 4C is IEC main peak % data.

FIGS. 5A-D shows the results of stability data at 25° C. FIG. 5A is subvisible particles data, FIG. 5B is SEC monomer % data, FIG. 5C is IEC main peak % data, and FIG. 5D is CE-SDS (NR) intact peak % data.

FIGS. 6A-D shows the results of stability data in 5±3° C., FIG. 6A is subvisible particles data, FIG. 6B is SEC monomer % data, FIG. 6C is IEC main peak % data, and FIG. 6D is CE-SDS (NR) intact peak % data.

### DETAILED DESCRIPTION OF THE DISCLOSURE

### Definitions

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

Throughout this specification and the claims which follow, unless the context requires otherwise, the word “comprise,” and variations such as “comprises” and “comprising”, will be understood to imply the inclusion of a stated amino acid sequence, DNA sequence, step or group thereof, but not the exclusion of any other amino acid sequence, DNA sequence, step. When used herein the term “comprising” can be substituted with the term “containing,” “including” or sometimes “having.”

The terms “administration,” “administering,” “treating,” and “treatment” herein, when applied to an animal, human, experimental subject, cell, tissue, organ, or biological fluid, means contact of an exogenous pharmaceutical, therapeutic, diagnostic agent, or antibody formulation to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both.

The “subject” as used herein is a mammal, e.g., a rodent or a primate, preferably a higher primate, e.g., a human (e.g., a patient having, or at risk of having, a disorder described herein).

The term “therapeutically effective amount” as herein used, refers to the amount of an anti-TIGIT antibody that, when administered to a subject for treating a disease, or at least one of the clinical symptoms of a disease or disorder, is sufficient to affect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition. In some embodiment of present disclosure, the subject is a human.

The term “antibody” herein is used in the broadest sense and specifically covers antibodies (including full length monoclonal antibodies) and antibody fragments so long as they recognize antigen, e.g., TIGIT. An antibody is usually monospecific, but may also be described as idiospecific, heterospecific, or polyspecific. Antibody molecules bind by means of specific binding sites to specific antigenic determinants or epitopes on antigens.

The term “monoclonal antibody” or “mAb” or “Mab” herein means a population of substantially homogeneous antibodies, i.e., the antibody molecules comprised in the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) may be obtained by methods known to those skilled in the art. See, for example Kohler G et al., Nature 1975 256:495-497; U.S. Pat. No. 4,376,110; Ausubel F M et al., CURRENT PROTOCOLS IN MOLECULAR BIOLOGY 1992; Harlow E et al., ANTIBODIES: A LABORATORY MANUAL, Cold spring Harbor Laboratory 1988; and Colligan J E et al., CURRENT PROTOCOLS IN IMMUNOLOGY 1993. The monoclonal antibodies disclosed herein may be of any immunoglobulin class including IgG, IgM, IgD, IgE, IgA, and any subclass thereof. A hybridoma producing a monoclonal antibody can be cultivated in vitro or in vivo. High titers of monoclonal antibodies can be obtained by in vivo production where cells from the individual hybridomas are injected intraperitoneally into mice, such as pristine-primed Balb/c mice to produce ascites fluid containing high concentrations of the desired monoclonal antibodies. Monoclonal antibodies of isotype IgM or IgG may be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains sequentially comprise FR-1 (or FR1), CDR-1 (or CDR1), FR-2 (FR2), CDR-2 (CDR2), FR-3 (or FR3), CDR-3 (CDR3), and FR-4 (or FR4). The assignment of amino acids to each domain is, generally, in accordance with the definitions of Sequences of Proteins of Immunological Interest, Kabat, et al., National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al, (1987) J Mol. Biol. 196:901-917 or Chothia, et al., (1989) Nature 342:878-883.

The term “hypervariable region” means the amino acid residues of an antibody that are responsible for antigen-binding. The hypervariable region comprises amino acid residues from a “CDR” (i.e., VL-CDR1, VL-CDR2 and VL-CDR3 in the light chain variable domain and VH-CDR1, VH-CDR2 and VH-CDR3 in the heavy chain variable domain). Sec, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196:901-917 (defining the CDR regions of an antibody by structure). The term “framework” or “FR” residues mean those variable domain residues other than the hypervariable region residues defined herein as CDR residues.

Unless otherwise indicated, “antibody fragment” or “antigen-binding fragment” means antigen binding fragments of antibodies, i.e., antibody fragments that retain the ability to bind specifically to the antigen bound by the full-length antibody, e.g., fragments that retain one or more CDR regions. Examples of antigen binding fragments include, but not limited to; Fab, Fab′, F(ab′)2, and Fv fragments; diabodies; linear antibodies; single-chain antibody molecules, e.g., single chain Fv (ScFv); nanobodies and multispecific antibodies formed from antibody fragments.

An antibody that binds to a specified target protein with specificity is also described as specifically binding to a specified target protein. This means the antibody exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies or binding fragments thereof, useful in the present invention will bind to the target protein with an affinity that is at least two-fold greater, preferably at least 10-times greater, more preferably at least 20-times greater, and most preferably at least 100-times greater than the affinity with non-target proteins. An antibody herein is said to bind specifically to a polypeptide comprising a given amino acid sequence.

The term “human antibody” herein means an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” means an antibody that comprises only mouse or rat immunoglobulin protein sequences, respectively.

The term “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The antibody of the present application has potential therapeutic uses in treating cancer. The term “cancer” or “tumor” herein means or describes the physiological condition in mammals that is typically characterized by unregulated cell growth. Examples of cancer include but are not limited to, lung cancer (including small-cell lung cancer, or non-small cell lung cancer), adrenal cancer, liver cancer, stomach cancer, cervical cancer, melanoma, renal cancer, breast cancer, colorectal cancer, leukemia, bladder cancer, bone cancer, brain cancer, endometrial cancer, head and neck cancer, lymphoma, ovarian cancer, skin cancer, thyroid tumor, or metastatic lesion of the cancer.

Further, the antibody of the present application has potential therapeutic uses in controlling viral infections and other human diseases that are mechanistically involved in immune tolerance or “exhaustion.” In the context of the present application, the term “exhaustion” refers to a process which leads to a depleted ability of immune cells to respond to a cancer or a chronic viral infection.

### Anti-TIGIT Antibody

The present disclosure provides for anti-TIGIT antibodies and formulations thereof. For example, Ociperlimab (BGB-A1217), is an anti-TIGIT antibody disclosed in PCT Patent No. WO2019/129261 with sequences provided in Table 1 below.

TABLE 1
Ociperlimab (BGB-A1217) is disclosed in WO2019/129261.
Ociperlimab

| SEQ ID | | |
|---|---|---|
| NO: | Domain | Sequence |
| HCDR1 | SEQ ID NO: 1 | DYYMY |
| HCDR2 | SEQ ID NO: 2 | YITKGGGSTYYPDSVKG |
| HCDR3 | SEQ ID NO: 3 | QTNYDFTMDY |
| LCDR1 | SEQ ID NO: 4 | KASQDVGTSVA |
| LCDR2 | SEQ ID NO: 5 | WASARHT |
| LCDR3 | SEQ ID NO: 6 | QQYSSYPLT |
| VH | SEQ ID NO: 7 | EVQLVESGGGLVQPGGSLRLSCAASGFTFSDYYMYWVRQAPG |
| | | KGLEWVAYITKGGGSTYYPDSVKGRFTISRDNAKNTLYLQMN |
| | | SLRAEDTAVYYCARQTNYDFTMDYWGQGTLVTVSS |
| VL | SEQ ID NO: 8 | EIVMTQSPATLSVSPGERATLSCKASQDVGTSVAWYQQKPGQA |
| | | PRLLIYWASARHTGIPARFSGSGSGTEFTLTISSLQSEDFAVYYC |
| | | QQYSSYPLTFGGGTKVEIK |
| Heavy chain | SEQ ID NO: 9 | EVQLVESGGGLVQPGGSLRLSCAASGFTFSDYYMYWVRQAPG |
| | | KGLEWVAYITKGGGSTYYPDSVKGRFTISRDNAKNTLYLQMN |
| | | SLRAEDTAVYYCARQTNYDFTMDYWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISR |
| | | TPEVTCVVVDVSHEDPEVKFNWYVDGVEVHNAKTKPREEQY |
| | | NSTYRVVSVLTVLHQDWLNGKEYKCKVSNKALPAPIEKTISKA |
| | | KGQPREPQVYTLPPSREEMTKNQVSLTCLVKGFYPSDIAVEWE |
| | | SNGQPENNYKTTPPVLDSDGSFFLYSKLTVDKSRWQQGNVFS |
| | | CSVMHEALHNHYTQKSLSLSPGK |
| light chain | SEQ ID NO: 10 | EIVMTQSPATLSVSPGERATLSCKASQDVGTSVAWYQQKPGQA |
| | | PRLLIYWASARHTGIPARFSGSGSGTEFTLTISSLQSEDFAVYYC |
| | | QQYSSYPLTFGGGTKVEIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| IgG1 WT | SEQ ID NO: 11 | ASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKKVEPKSCDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALPAP |
| | | IEKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |

### Methods of Treatment

The antibodies or antigen-binding fragments of the present disclosure are useful in a variety of applications including, but not limited to, methods for the treatment of a TIGIT-associated disorder or disease. In one aspect, the TIGIT-associated disorder or disease is cancer.

In one aspect, the present disclosure provides a method of treating cancer. In certain aspects, the method comprises administering to a patient in need an effective amount of an anti-TIGIT antibody or antigen-binding fragment. The cancer can include, without limitation, lung cancer (including small-cell lung cancer, or non-small cell lung cancer), adrenal cancer, liver cancer, stomach cancer, cervical cancer, melanoma, renal cancer, breast cancer, colorectal cancer, leukemia, bladder cancer, bone cancer, brain cancer, endometrial cancer, head and neck cancer, lymphoma, ovarian cancer, skin cancer, thyroid tumor, or metastatic lesion of the cancer.

An antibody or antigen-binding fragment of the invention can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Anti-TIGIT antibodies or antigen-binding fragments can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or antigen-binding fragment of the invention will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 200 mg-2400 mg of antibody can be an initial dosage for administration to the patient, whether, for example, by one or more separate administrations, or by continuous infusion. One typical daily dosage might range from about 200 mg-2400 mg of antibody or more, depending on the factors mentioned above. For repeated administrations over several days or longer, depending on the condition, the treatment would generally be sustained until a desired suppression of disease symptoms occurs. Such doses can be administered intermittently, e.g., every week, every two weeks or every three weeks. An initial higher loading dose, followed by one or more lower doses can be administered. However, other dosage regimens can be useful. The progress of this therapy is easily monitored by conventional techniques and assays.

### Pharmaceutical Compositions and Formulations

Also provided are compositions, including pharmaceutical formulations, comprising an anti-TIGIT antibody or antigen-binding fragment thereof, or polynucleotides comprising sequences encoding an anti-TIGIT antibody or antigen-binding fragment. In certain embodiments, compositions comprise one or more antibodies or antigen-binding fragments that bind to TIGIT, or one or more polynucleotides comprising sequences encoding one or more antibodies or antigen-binding fragments that bind to TIGIT. These compositions can further comprise suitable carriers, such as pharmaceutically acceptable excipients including buffers, which are well known in the art.

Pharmaceutical formulations of an anti-TIGIT antibody or antigen-binding fragment as described herein are prepared by mixing such antibody or antigen-binding fragment having the desired degree of purity with one or more optional pharmaceutically acceptable carriers, in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Sustained-release preparations can be prepared. Suitable examples of sustained-release preparations include semipermeable matrices of solid hydrophobic polymers containing the antibody, which matrices are in the form of shaped articles, e.g., films, or microcapsules.

The formulations to be used for in vivo administration are generally sterile. Sterility can be readily accomplished, e.g., by filtration through sterile filtration membranes. CL EXAMPLES

The examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

### Analytical Methods

This methods section provides a summary of the methods used in the following Examples 1-4.

### SEC-HPLC

Formation of soluble aggregates and fragments is analyzed by size exclusion chromatography (SEC) on a Waters™ HPLC system. Protein is separated based on molecular size on a TSK gel Super SW™ mAb HR, 7.8×300 mm column maintained at 37±5° C. using an isocratic gradient. Molecular weight species are eluted and detected by UV absorption at 215 nm. The distribution of aggregates, monomer and fragments are quantitated via the peak areas for standards and samples.

### IEC-HPLC

The Ion exchange chromatography (IEC) IEC-HPLC method was used to evaluate the charge variants by cation exchange chromatography. The HPLC system (Waters) was equipped with a Thermo MabPac SCX-10™ analytical column (4×250 mm) at 37±5° C. Gradient NaCl elution was performed at a constant flow rate of 1.0 mL/min and UV signals were obtained at 280 nm. Peaks in the IEC-HPLC chromatogram were integrated, and percentage peak areas of each peak were calculated.

### CE-SDS (NR).

The purity of sample is determined using PA800 Plus™ (Beckman) by a capillary gel electrophoresis (CE) method. Samples are denatured with sodium dodecyl sulphate (SDS) and separated based on size in a capillary filled with a gel that acts as a sieving medium. In non-reduced (NR) samples, an alkylating agent, N-Ethylmaleimide (NEM), is added to avoid any fragmentation induced by sample preparation and to ensure that the main IgG peak remains intact. Samples are injected electrokinetically and the mobilized proteins are detected by UV absorbance at 200 nm using a UV detector. The reportable value for non-reduced samples is the time corrected area percent (TCA) % of the IgG main peak.

### Protein Concentration

Protein concentrations are determined at UV 280 nm.

### Tm and Tagg

Determining stability was performed using Uncle™ (Unchained Labs) which combines 3 different measurement modes-fluorescence, Static Light Scattering (SLS) and Dynamic Light Scattering (DLS). SLS and intrinsic fluorescence were conducted to determine the temperature of on-set aggregation (Tagg) and melting temperature (Tm) of formulations, respectively.

### Turbidity

NEPHELOstar plus™ with wavelength 635 nm of laser light was used for turbidity testing. Samples were put into 96 well plate and read turbidity by NEPHELOstar plus™.

### Visible Particles

Visible particles were examined against a black background and a white background using white fluorescent light at about 2000 lux. The vial under inspection was gently swirled and inspected for no less than 10 seconds against each of the backgrounds. The total inspection time was about 20 seconds.

Subvisible Particles Subvisible particles were analyzed using micro-flow imaging (MFI, Micro-Flow Imaging™ ProteinSimple). A water flush was performed before each sample analysis to ensure the background counts were appropriate for testing. The average cumulative counts per mL were reported.

### Example 1: Impact of pH and Buffer on the Stability of Anti-TIGIT Antibody Formulations

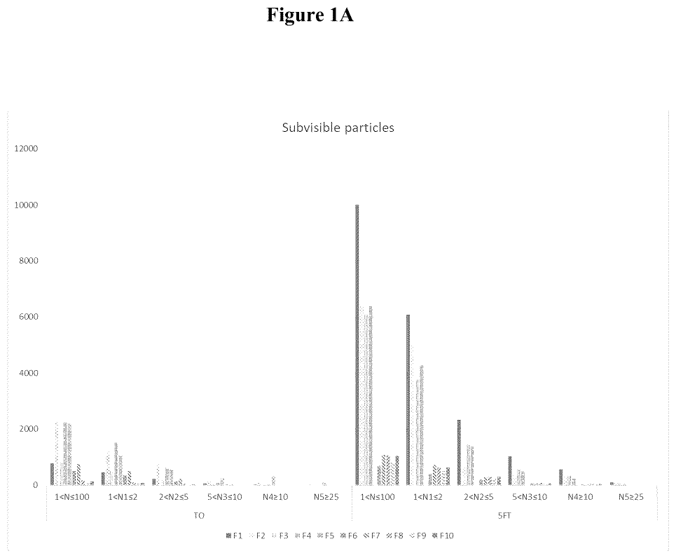

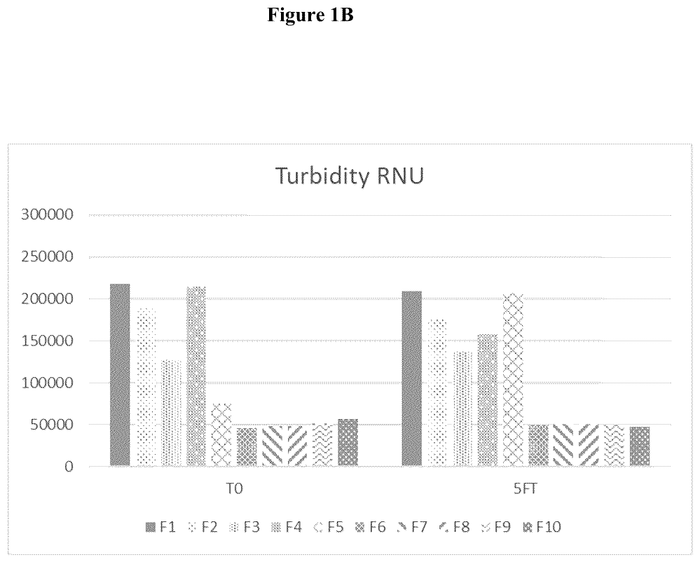

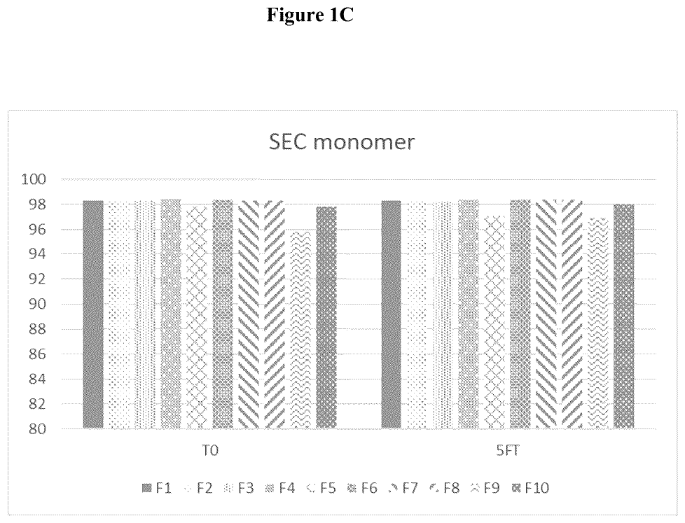

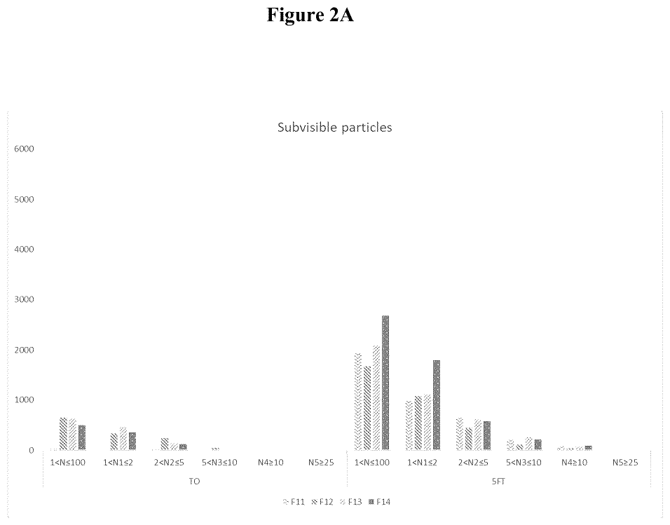

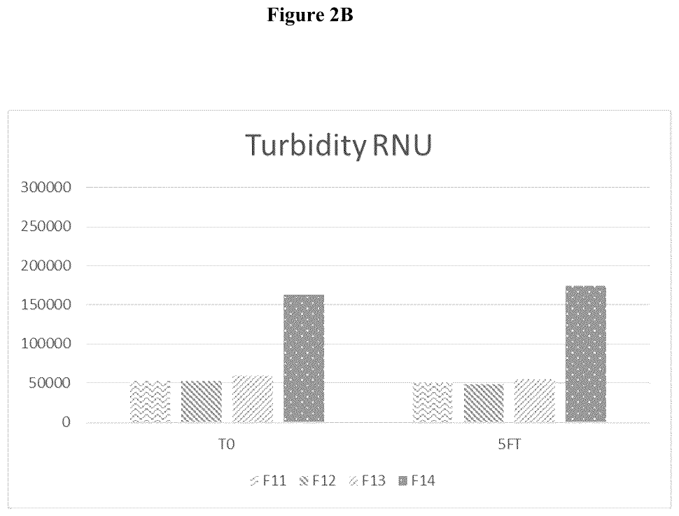

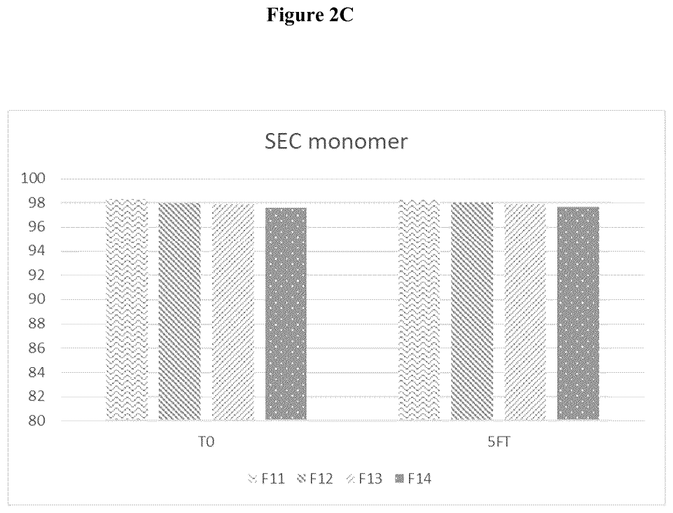

In order to determine the stability of the antibody in different pH and buffer, Ociperlimab was dialyzed into different buffers with 10 kDa MWCO dialysis cassettes and prepared into the formulations listed as Table 2. These formulations were filtered by Millex™ GP 0.22 mm PES 33 mm filter and filled into 2 mL ready-to-use glass vials. To evaluate the effect on antibody stability, these antibody samples were tested Tm and Tagg with Uncle™ from Unchained Labs and the data is shown in Table 2. In addition, these antibody samples were placed in freeze-thaw for five cycles (noted as “5 FT” in FIG. 2). As a control, these antibody samples were assayed at its initial time point, noted as TO in FIGS. 1A-C. Subvisible particles, turbidity, and purity by SEC-HPLC were measured for all samples. Results are shown graphically in FIGS. 1A-C.

TABLE 2
Tm, Tagg of Anti-TIGIT Antibody in Different pH and Buffer

| | | Protein | | | |
|---|---|---|---|---|---|
| | | concentration | | | |
| NO. | Formulation | (mg/ml) | Tm1(° C.) | Tm2(° C.) | Tagg(° C.) |
| F1 | 20 mM succinic acid- sodium | 10 | 65.82 | 78.27 | 74.92 |
| | hydroxide, pH 5.0 | | | | |
| F2 | 15 mM citric acid- sodium citrate, | 10 | 66.9 | 74.90 | 71.43 |
| | pH 5.5 | | | | |
| F3 | 30 mM acetic acid- sodium | 10 | 67.91 | 77.07 | 73.98 |
| | acetate, pH 5.5 | | | | |
| F4 | 20 mM Histidine-Histidine HCl, | 10 | 67.78 | 82.00 | 75.25 |
| | pH 6.0 | | | | |
| F5 | 20 mM sodium dihydrogen | 10 | 70.83 | 82.33 | 71.68 |
| | phosphate-disodium hydrogen | | | | |
| | phosphate, pH 7.0 | | | | |
| F6 | 20 mM succinic acid- sodium | 10 | 67.05 | 79.49 | 76.02 |
| | hydroxide, 240 mM sucrose, 0.2 | | | | |
| | mg/ml polysorbate 80, pH 5.0 | | | | |
| F7 | 15 mM citric acid- sodium | 10 | 68.07 | 75.37 | 72.99 |
| | citrate, 240 mM sucrose, 0.2 mg/ml | | | | |
| | polysorbate 80, pH5.5 | | | | |
| F8 | 30 mM acetic acid- sodium | 10 | 68.21 | 78.61 | 75.39 |
| | acetate, 240 mM sucrose, 0.2 | | | | |
| | mg/ml polysorbate 80, pH 5.5 | | | | |
| F9 | 20 mM Histidine-Histidine HCl, | 10 | 67.97 | 83.67 | 75.17 |
| | 240 mM sucrose, 0.2 mg/ml | | | | |
| | polysorbate 80, pH 6.0 | | | | |
| F10 | 20 mM sodium dihydrogen | 10 | N/A | 75.37 | 73.02 |
| | phosphate-disodium hydrogen | | | | |
| | phosphate, 240 mM sucrose, 0.2 | | | | |
| | mg/ml polysorbate 80, pH 7.0 | | | | |

Note:
N/A meant no Tm1 was found in the test.

From Table 2, all the formulations had higher than 65° C. of Tm1, higher than 75° C. of Tm2 and higher than 70° C. of Tagg, which were good indications of stability. In the formulations without stabilizer and surfactant, the subvisible particles and turbidity increased after five cycles of freeze-thaw, while in the antibody formulation with stabilizer and surfactant there was no obvious increase. All these formulations showed no obvious change and not less than 95% in SEC monomer after five cycles of freeze-thaw. Taken together, the subvisible particles data (FIG. 1A), and the turbidity data (FIG. 1B), and the SEC-HPLC data (FIG. 1C) demonstrated that Ociperlimab antibody was stable in formulations in different buffers with stabilizer and surfactant at pH 5.0-7.0.

### Example 2: Effect of Protein Concentration on the Stability of Anti-TIGIT Antibody Formulations

To investigate the impact of Ociperlimab antibody protein concentration on the stability of Ociperlimab antibody formulation, a stress study in freeze-thaw conditions was performed. For F11-F13, a high concentration of Ociperlimab was generated using 30 kDa Amicon Ultra™ centrifugal filters to create a concentrated stock solution. This stock solution was dialyzed into 30 mM acetic acid-sodium acetate buffer and then used to produce the formulations listed in Table 3. For F14, a concentrated stock solution of Ociperlimab was generated by using 30 kDa Amicon Ultra™ centrifugal filters and then dialyzed into 20 mM histidine-histidine HCl buffer and then adjusted to the F14 formulation found in Table 3. Each of the formulated solutions was filtered using a 0.22 mm PES syringe filter and placed in freeze-thaw stress conditions. To evaluate the effect on antibody stability, these antibody samples were tested Tm and Tagg with Uncle™ from Unchained Labs and data is shown in Table 3.

TABLE 3
Tm and Tagg of Anti-TIGIT Antibody with Different Protein Concentration

| | | Protein | | | |
|---|---|---|---|---|---|
| | | concentration | | | |
| NO. | Formulation | (mg/ml) | Tm1(° C.) | Tm2(° C.) | Tagg(° C.) |
| F11 | 30 mM acetic acid- sodium | 10 | 70.66 | 80.73 | 77.58 |
| | acetate, 240 mM sucrose, 0.2 | | | | |
| | mg/ml polysorbate 80, pH 5.5 | | | | |
| F12 | 30 mM acetic acid- sodium | 20 | 70.47 | 79.07 | 76.52 |
| | acetate, 240 mM sucrose, 0.2 | | | | |
| | mg/ml polysorbate 80, pH 5.5 | | | | |
| F13 | 30 mM acetic acid- sodium | 40 | N/A | 78.34 | 75.55 |
| | acetate, 240 mM sucrose, 0.2 | | | | |
| | mg/ml polysorbate 80, pH 5.5 | | | | |
| F14 | 20 mM histidine-histidine HCl, | 150 | 73.35 | 77.93 | 72.04 |
| | 70 mM NaCl, 80 mM trehalose, 0.8 | | | | |
| | mg/ml polysorbate 20, pH 6.0 | | | | |

Note:
N/A meant no Tm1 was found in the test.

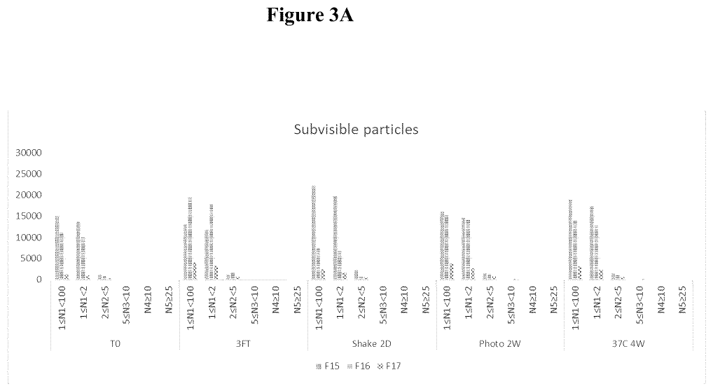

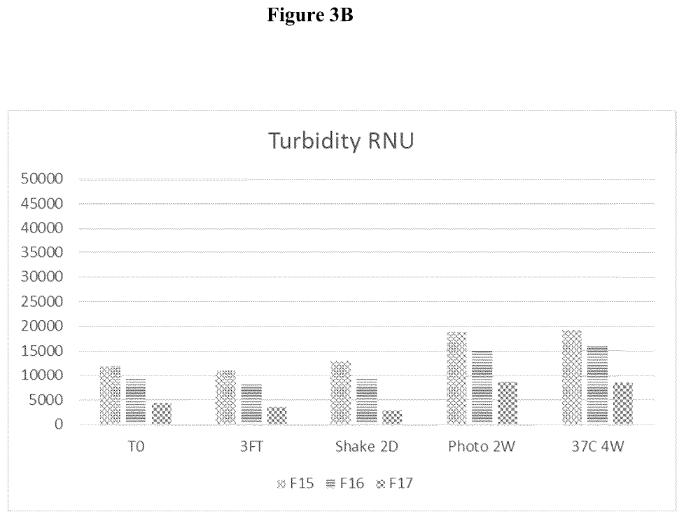

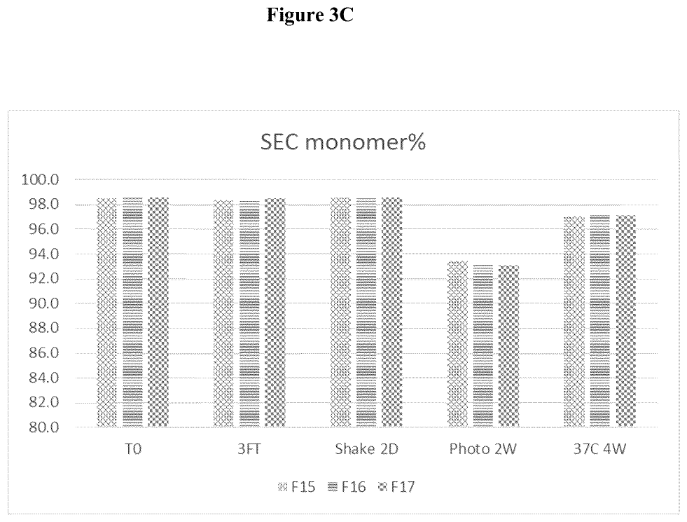

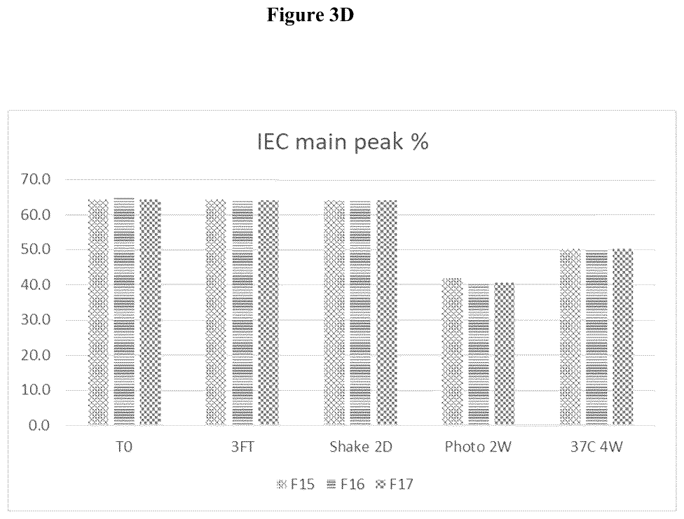

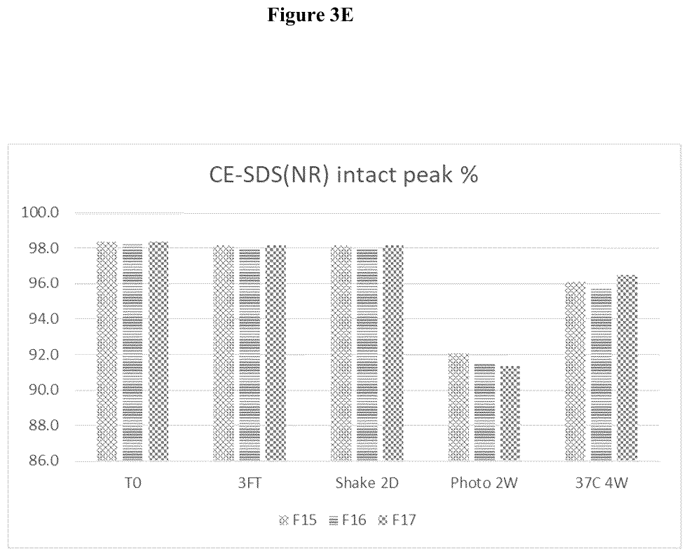

From Table 3, all the formulations had higher than 65° C. of Tm1, higher than 75° C. of Tm2 and higher than 70° C. of Tagg, which were good indications of stability. In the freeze-thaw stability study, subvisible particles slightly increased comparing with zero timepoint (T0) samples, especially for the subvisible particles smaller than 10 μm. However, the total count of subvisible particles remained low, especially for the subvisible particles larger than 10 μm. From the turbidity data, the turbidity increased with increased protein concentration, but there was no obvious change after five cycles of freeze-thaw five cycles, indicating the Ociperlimab antibody was stable for these formulations. From SEC-HPLC data, there was no obvious change in SEC monomer after five cycles of freeze-thaw and all the samples had greater than 95% SEC monomer, indicating these formulations with Ociperlimab antibody concentrations from 10-150 mg/ml were not prone to aggregate or form particles. Combining the data shown in FIGS. 3A-C, all of the formulations tested were stable in a wide range of Ociperlimab antibody protein concentrations ranging from 10 mg/ml to 150 mg/ml.

### Example 3 Effect of Surfactants on the Stability of Anti-TIGIT Antibody Formulations

This experiment investigated the effect of different surfactants on the stability of Ociperlimab antibody formulations. In this study, Ociperlimab was buffer exchanged into 20 mM histidine-histidine HCl buffer (pH 5.8) by dialysis, and then adjusted with different surfactants to form the formulations listed in Table 4. Each of the formulated solutions was filtered using a 0.22 mm PES syringe filter and placed in stress conditions.

TABLE 4
Formulation Compositions with different Surfactants

| No. | Formulation Composition |
|---|---|
| F15 | 20 mg/ml antibody protein concentration, 20 mM histidine- |
| | histidine HCl, 240 mM trehalose, 0.5 mg/ml polysorbate 20, pH |
| | 5.8 |
| F16 | 20 mg/ml antibody protein concentration, 20 mM histidine- |
| | histidine HCl, 240 mM trehalose, 0.5 mg/ml polysorbate 80, pH |
| | 5.8 |
| F17 | 20 mg/ml antibody protein concentration, 20 mM histidine- |
| | histidine HCl, 240 mM trehalose, 0.5 mg/ml poloxamer188, pH 5.8 |

A freeze-thaw study was performed by subjecting the formulations to three cycles of freezing at −70° C. and then thawing at ambient temperature (noted as “3 FT” in FIGS. 3A-E). In order to study the high temperature stability and photostability of formulations, Ociperlimab antibody formulations were either stored in a 37° C. stability chamber for 4 weeks (noted as “37C4W” in tables and graphs) or were placed in a photostability chamber for 2 weeks with no less than 1.2 million lux hours illumination (noted as “photo 2W” in Figures). Mechanical stress study was performed by subjecting formulations to shaking conditions using 800 rpm for 2 days (noted as “shake 2D” in Figures). The formulations were evaluated by subvisible particles, turbidity, SEC (purity), IEC (charge profiles) and CE-SDS (NR) (purity). As a control, the samples were analyzed at its initial time point (noted as TO in the Figures).

As demonstrated in FIG. 3A-B, Ociperlimab formulations F15, F16 and F17 showed no obvious increase in subvisible particles and turbidity under the conditions of three cycles of freeze-thaw, shaking for 2 days, photostability 2 weeks and holding at 37° C. for 4 weeks. These data indicate that Ociperlimab in these formulations was not prone to aggregate or to form particles. Note that F17, an antibody formulation with poloxamer 188 showed less subvisible particles and turbidity than those formulations containing polysorbate 20 and polysorbate 80. As shown in FIG. 3 C-E, formulations F15, F16 and F17 showed no obvious change in SEC, IEC, CE-SDS (NR) under the condition of three cycles of freeze-thaw, shaking for 2 days and holding at 37° C. 4 weeks. Under the conditions of 2 weeks stress testing under photostability conditions, the F15, F16 and F17 formulations showed no obvious change in the purity of SEC, IEC, CE-SDS (NR) when compared to the initial time point (TO). Taking into account these stability data, polysorbate 20, polysorbate 80 and poloxamer 188, were contributing to the Ociperlimab antibody stability in the formulation.

### Example 4. Stability of Anti-TIGIT Antibody Formulations

The stability of the Ociperlimab antibody was evaluated in the various formulations listed in Table 5. All formulations were prepared in 20 mM histidine-histidine HCl buffers with a pH range from 5.2 to 6.2. Trehalose at a concentration of 240 mM was used as stabilizer and tonicity modifier. The surfactant was polysorbate 20 held at a concentration of 0.2 mg/ml. The Ociperlimab antibody was buffer exchanged into 20 mM histidine-histidine HCl buffers with different pH (pH 5.2, 5.5, 5.8, 6.0, 6.2) by dialysis to generate Ociperlimab stock solutions at the intended pH and an Ociperlimab antibody concentration of 20 mg/ml was used. Each of the formulated solutions was filtered using a 0.22 mm PES syringe filter and placed in stability studies as showed in Table 6.

TABLE 5
Formulation Compositions Used in Stability Studies

| No. | Formulation Composition |
|---|---|
| F18 | 20 mg/ml protein concentration, 20 mM Histidine-Histidine HCl, |
| | 240 mM Trehalose, 0.2 mg/ml polysorbate 20, pH 5.2 |
| F19 | 20 mg/ml protein concentration, 20 mM Histidine-Histidine HCl, |
| | 240 mM Trehalose, 0.2 mg/ml polysorbate 20, pH 5.5 |
| F20 | 20 mg/ml protein concentration, 20 mM Histidine-Histidine HCl, |
| | 240 mM Trehalose, 0.2 mg/ml polysorbate 20, pH 5.8 |
| F21 | 20 mg/ml protein concentration, 20 mM Histidine-Histidine HCl, |
| | 240 mM Trehalose, 0.2 mg/ml polysorbate 20, pH 6.0 |
| F22 | 20 mg/ml protein concentration, 20 mM Histidine-Histidine HCl, |
| | 240 mM Trehalose, 0.2 mg/ml polysorbate 20, pH 6.2 |

TABLE 6
Formulation Stability Studies Scheme

| Study Conditions | | Test Items |
|---|---|---|
| High temperature | Samples were placed in a 40° C. | Visible particles |
| (40° C.) study | stability chamber for 2 weeks | Subvisible particles |
| Photostability study | Total illumination no less than 1.2 | Purity (SEC) |
| | million lux hours | Charge variants (IEC) |
| Freeze-thaw study | Six cycles of freezing at −70° C. and | |
| | thawing at ambient temperature. | |
| Accelerated | Samples were placed in a 25° C. | Visible particles |
| stability (25° C.) | stability chamber for 6 months | Subvisible particles |
| study | | Purity (SEC and CE-SDS(NR)) |
| Long-term stability | Samples were placed in a 5 ± 3° C. | Charge variants (IEC) |
| (5 ± 3° C.) study | stability chamber for 24 months | |

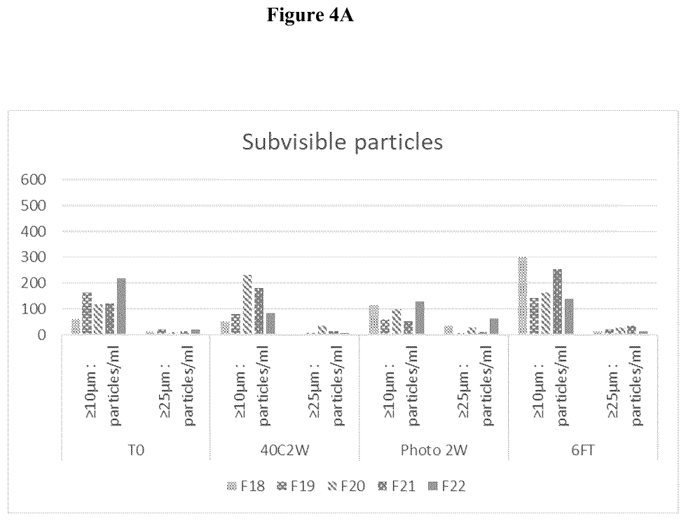

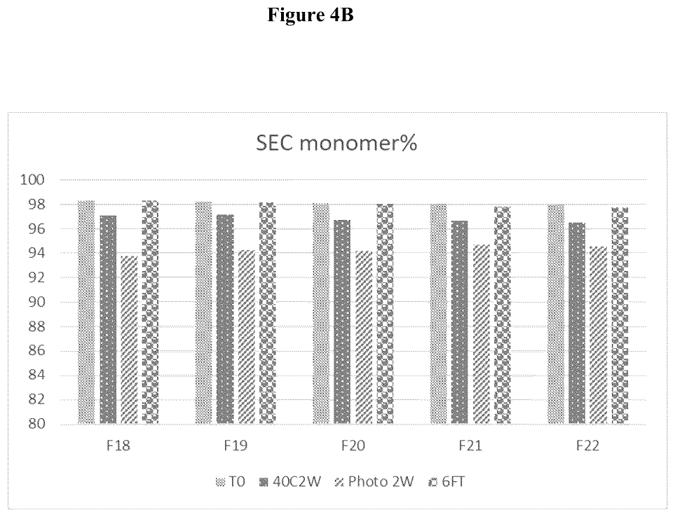

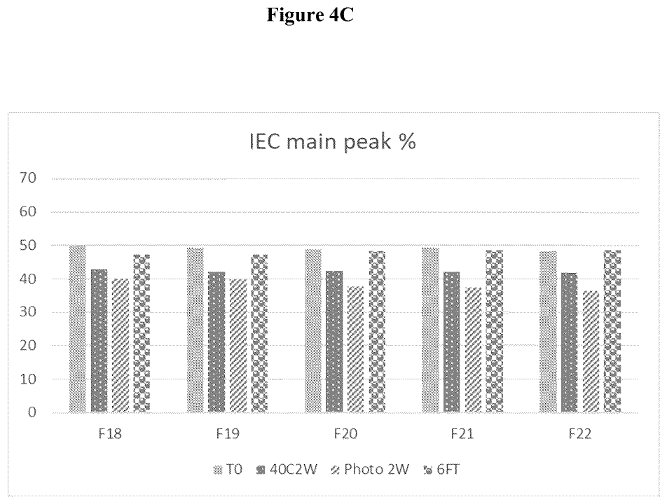

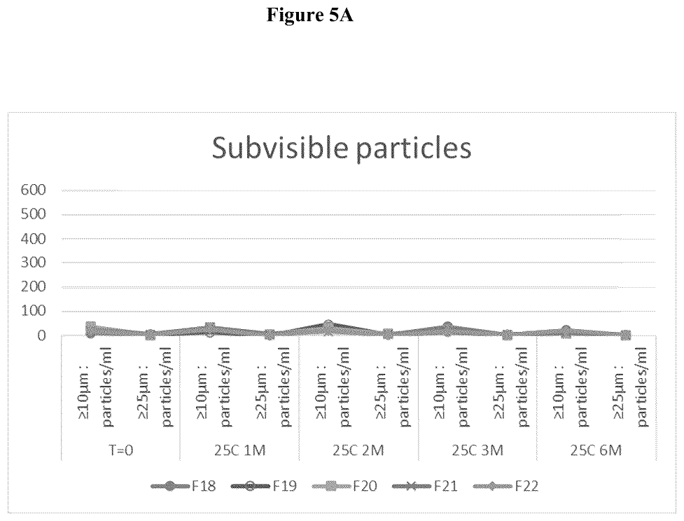

A freeze-thaw study was performed by subjecting the samples to six cycles of freezing at −70° C. and thawing at ambient temperature (noted as “6 FT” in Table 7). In order to determine the effect of high temperature stability and photostability of formulations, samples were either stored in a 40° C. stability chamber for 2 weeks (noted as “40C 2W” in tables and graphs) or were placed in a photostability chamber for 2 weeks with no less than 1.2 million lux hours illumination (noted as “photo 2W” in Table 7). The accelerated stability study in 25° C. and the long-term stability study in 5±3° C. were also investigated (noted as “25C” and “5C” in Tables 8 and 9). The formulations were evaluated by visible particles, subvisible particles, SEC (purity), IEC (charge profiles) and CE-SDS (NR) (purity). As a control, the samples were analyzed at its initial time point (noted as TO). These results were provided in Tables 7-9 and FIGS. 4-6.

From the visible particles data in Table 7, all the formulations were clear in six cycles of freeze-thaw, and for formulations tested at 40° C. for 2 weeks and for photostability, no visible particles were observed. From the subvisible particles data in FIG. 4A, all the formulations were stable in six cycles of freeze-thaw, 40° C. for 2 weeks and photostability, with no obvious increase in subvisible particles. From the SEC data in FIG. 4 B, all the formulations were stable under six cycles of freeze-thaw, and monomer purity was slightly decreased in 40° C. for 2 weeks and slight decrease in photostability. From the IEC data in FIG. 4 C, all the formulations were stable under six cycles of freeze-thaw, and main peak slightly decreased in 40° C. for 2 weeks and a slight decrease in photostability.

TABLE 7
Visible particles assay for Formulation F18, F19, F20,
F21 and F22 under the specified stress conditions

| Formulation | T0 | 40 C. 2 W | Photo 2W | 6FT |
|---|---|---|---|---|
| F18 | clear | clear | clear | clear |
| F19 | clear | clear | clear | clear |
| F20 | clear | clear | clear | clear |
| F21 | clear | clear | clear | clear |
| F22 | clear | clear | clear | clear |

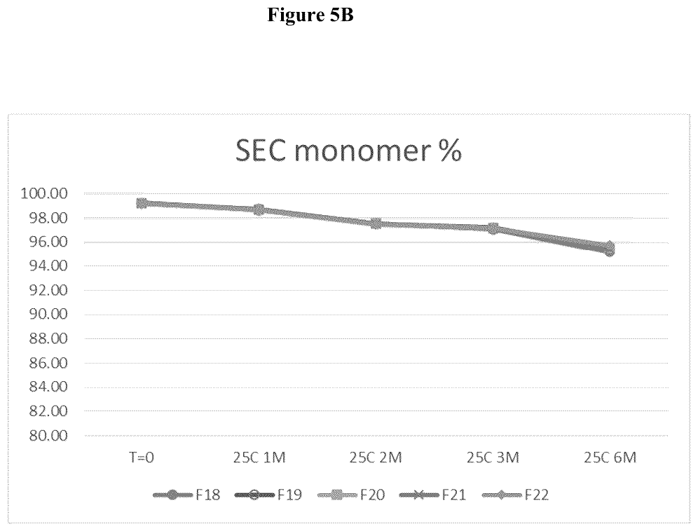

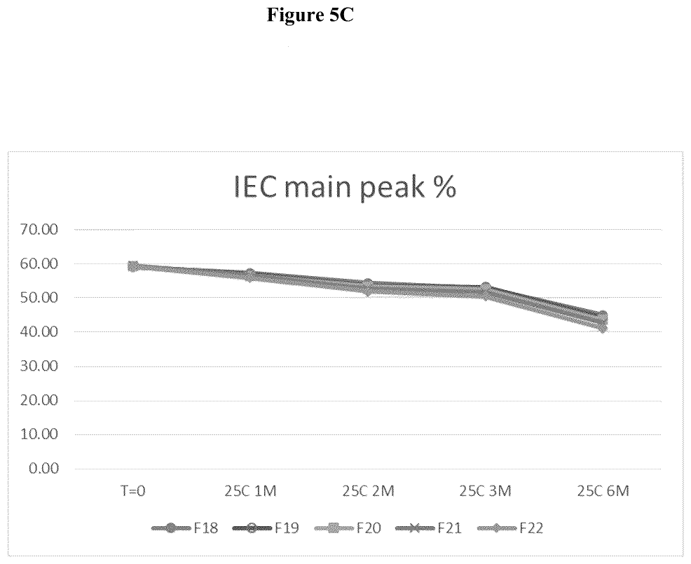

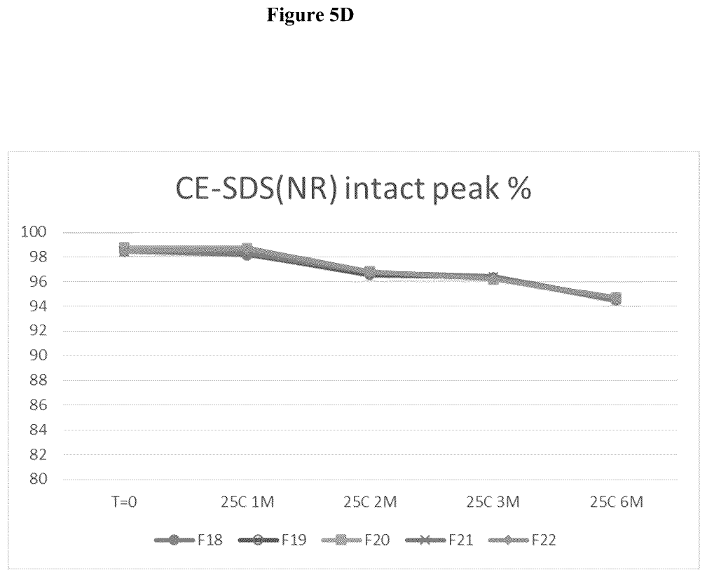

The stability data for Ociperlimab formulations kept at 25° C. are shown in Table 8 and FIG. 5. All the formulations were clear when stored at 25° C. for 6 months, and no visible particles were observed. From the subvisible particles data in FIG. 5A, all the formulations were stable in in 25° C. for 6 months, with no increase in subvisible particles. From the SEC data in FIG. 5 B, the monomer % decreased slightly over time, but remained over 95% for the longest endpoint of 25° C. for 6 months. This indicates that the formulation would pass the 95% SEC standard (the general specification for monoclonal antibodies). From the IEC data in FIG. 5 C, the main peak % deceased over time, but remained over 40% under the conditions of 25° C. for 6 months. From the CE-SDS (NR) data in FIG. 5 D, the intact peak % deceased over time, but remained over 94% when measured at 25° C. for 6 months.

TABLE 8
Visible particles result for Formulation
F18, F19, F20, F21 and F22 in 25° C.

| Formulation | T = 0 | 25 C. 1 M | 25 C. 2 M | 25 C. 3 M | 25 C. 6 M |
|---|---|---|---|---|---|
| F18 | clear | clear | clear | clear | clear |
| F19 | clear | clear | clear | clear | clear |
| F20 | clear | clear | clear | clear | clear |
| F21 | clear | clear | clear | clear | clear |
| F22 | clear | clear | clear | clear | clear |

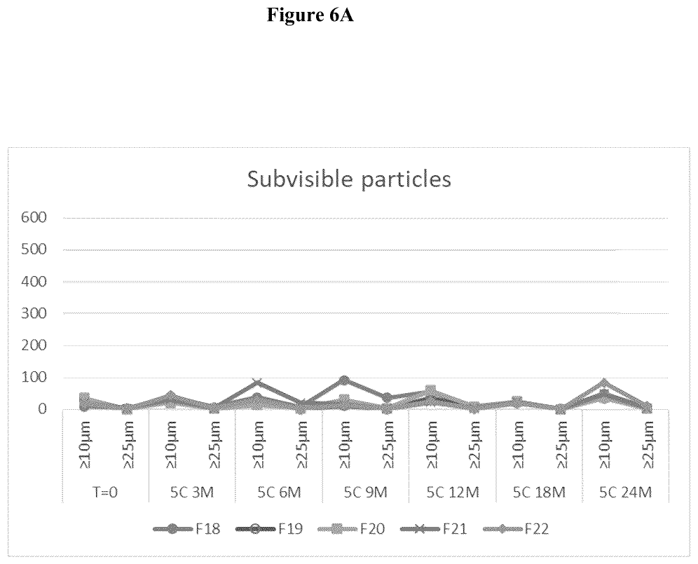

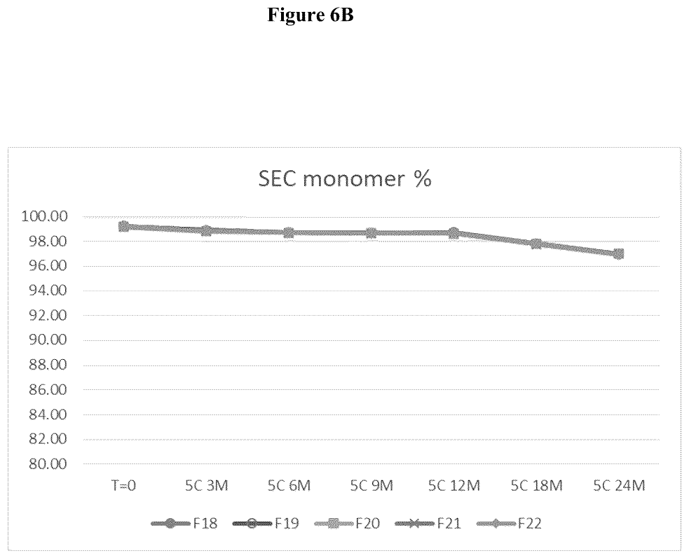

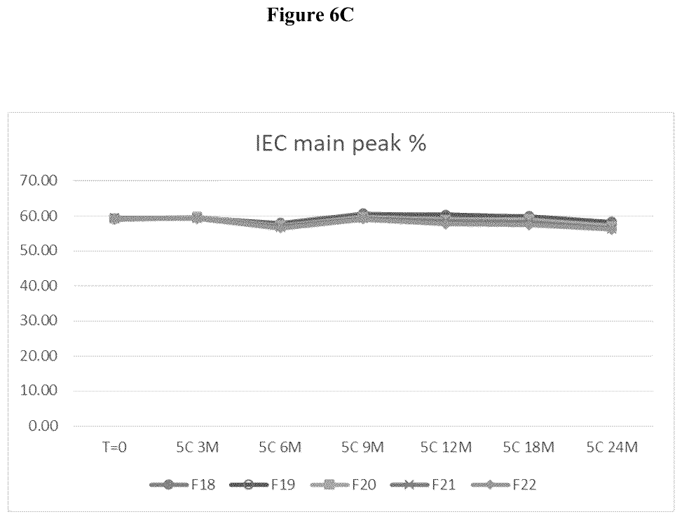

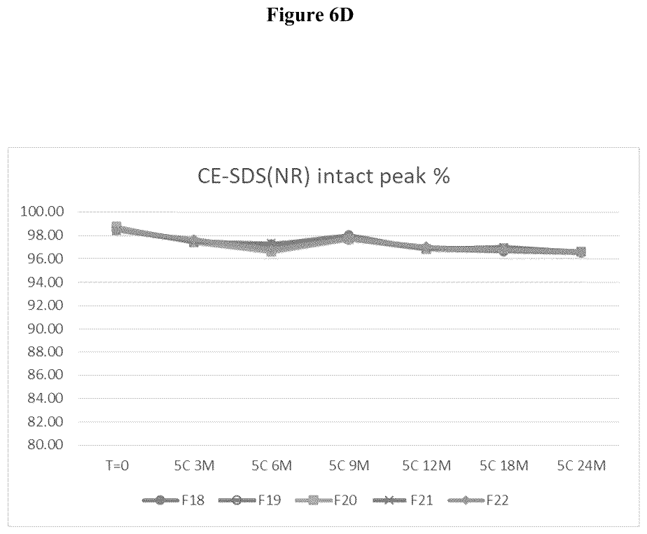

The long-term stability of Ociperlimab antibody formulations stored at 5±3° C. for various timepoints are shown in Table 9 and FIG. 6. All of the formulations were clear in 5±3° C. for 24 months, and no visible particles were observed. From the subvisible particles data in FIG. 6A, all formulations were stable in 5±3° C. for 24 months, with no increase in subvisible particles. From the SEC data shown in FIG. 6 B, the monomer % slightly decreased over time, but remained 97% at the 24 months timepoint in 5±3° C. Again, this indicates that the formulations disclosed herein would pass the 95% SEC standard (the general specification for monoclonal antibodies). From the IEC data in FIG. 6 C, the main peak % did not change with time in 5±3° C. for 24 months. From the CE-SDS (NR) data in FIG. 6 D, the intact peak % slightly deceased over time, but remained over 96% in 5±3° C. for 24 months.

TABLE 9
Visible particles result for Formulation F18, F19, F20, F21 and F22 in 5 ± 3° C.

| Formulation | T = 0 | 5 C. 3 M | 5 C. 6 M | 5 C. 9 M | 5 C. 12 M | 5 C. 18 M | 5 C. 24 M |
|---|---|---|---|---|---|---|---|
| F18 | clear | clear | clear | clear | clear | clear | clear |
| F19 | clear | clear | clear | clear | clear | clear | clear |
| F20 | clear | clear | clear | clear | clear | clear | clear |
| F21 | clear | clear | clear | clear | clear | clear | clear |
| F22 | clear | clear | clear | clear | clear | clear | clear |

In conclusion, these results demonstrated that Ociperlimab antibody formulations F18, F19, F20, F21 and F22 are stable at 5±3° C. and at 25° C. for 24 months and fall within acceptable parameters for clinical use.

1. A pharmaceutical formulation comprising:
i. about 5 mg/mL to about 200 mg/mL of an anti-TIGIT antibody, or antigen binding fragment thereof;
ii. about 5 mM to about 50 mM formulation buffer providing a pH of about 5.0 to about 7.0;
iii. about 30 mM to about 300 mM stabilizer;
iv. about 0.01 mg/ml to about 1 mg/ml non-ionic surfactant.

2. The formulation of claim 1, wherein the anti-TIGIT antibody or antigen binding fragment thereof, comprises a heavy chain variable region that comprises a HCDR1 (Heavy Chain Complementarity Determining Region 1) of SEQ ID NO: 1, a HCDR2 of SEQ ID NO: 2 and a HCDR3 of SEQ ID NO: 3 and a light chain variable region that comprises: a LCDR1 (Light Chain Complementarity Determining Region 1) of SEQ ID NO: 4, a LCDR2 of SEQ ID NO: 5 and a LCDR3 of SEQ ID NO: 6.

3. The formulation of claim 1, wherein the anti-TIGIT antibody or antigen binding fragment thereof, comprises SEQ ID NO:7 and SEQ ID NO:8.

4. The formulation of claim 1, wherein the formulation buffer is selected from the group consisting of histidine, acetate, citrate, succinate, phosphate, mixture of histidine and acetic acid, or mixture of histidine and citric acid.

5. The formulation of claim 4, wherein the formulation buffer is histidine, and wherein the concentration of the histidine buffer is 10 mM to 30 mM.

6. (canceled)

7. (canceled)

8. The formulation of claim 6 wherein the pH is a range of 5.2-6.2.

9. The formulation of claim 1, wherein the stabilizer is selected from the group consisting of trehalose, sucrose, sorbitol, mannitol, maltose, dextran, (2-hydroxypropyl)-b-cyclodextrin, sodium chloride, magnesium chloride, calcium chloride, sodium sulfate, sodium dihydrogen phosphate, or disodium hydrogen phosphate.

10. The formulation of claim 9, wherein the stabilizer is trehalose, and wherein the trehalose concentration is from 50 mM to 280 mM.

11. (canceled)

12. (canceled)

13. The formulation of claim 9, wherein the stabilizer is sucrose, and wherein the sucrose concentration is from 50 mM to 280 mM.

14. (canceled)

15. (canceled)

16. The formulation of claim 1, wherein the non-ionic surfactant is selected from the group consisting of polysorbate 20, polysorbate 80 or poloxamer188.

17. The formulation of claim 16, wherein the concentration of polysorbate 20 is from 0.1 mg/ml to 0.8 mg/ml.

18. The formulation of claim 17, wherein polysorbate 20 concentration is from 0.2 mg/ml to 0.6 mg/ml.

19. The formulation of claim 16, wherein the concentration of polysorbate 80 is from 0.1 mg/ml to 0.8 mg/ml.

20. (canceled)

21. The formulation of claim 16, wherein the concentration of poloxamer 188 is from 0.1 mg/ml to 0.8 mg/ml.

22. (canceled)

23. The formulation of claim 1, wherein the formulation comprises 30 mM acetic acid-sodium acetate, 240 mM sucrose, and 0.2 mg/ml polysorbate 80 with a pH of pH 5.5.

24. The formulation of claim 1, wherein the formulation comprises 20 mM Histidine-Histidine HCl, 240 mM trehalose and 0.2 mg/ml polysorbate 20, with a pH of pH 5.8.

25. The formulation of claim 1, wherein the formulation comprises 20 mM Histidine-Histidine HCl, 70 mM NaCl, 80 mM trehalose and 0.8 mg/ml polysorbate 20, with a pH of pH 6.0.

26. The formulation of claim 1, wherein the concentration of the anti-TIGIT antibody, or antigen binding fragment thereof is from about 10 mg/mL to 150 mg/mL.

27. A method of making an antibody formulation, the method comprising:
a. exchanging the anti-TIGIT antibody to about 5 mM to about 50 mM buffer providing a pH of about 5.0 to about 7.0;
b. concentrating the antibody formulation of (a) to an antibody concentration of about 5-200 mg/mL;
c. adding non-ionic surfactant to the antibody formulation of (c) to achieve an antibody formulation having a concentration of surfactant of no less than 0.01 mg/ml; and
d. adding stabilizer to the antibody to achieve an antibody formulation having a concentration of stabilizer no less than 30 mM, wherein the anti-TIGIT antibody comprises a heavy chain variable region that comprises a HCDR1 (Heavy Chain Complementarity Determining Region 1) of SEQ ID NO: 1, a HCDR2 of SEQ ID NO: 2 and a HCDR3 of SEQ ID NO: 3 and a light chain variable region that comprises: a LCDR1 (Light Chain Complementarity Determining Region 1) of SEQ ID NO: 4, a LCDR2 of SEQ ID NO: 5 and a LCDR3 of SEQ ID NO: 6.

28. (canceled)

29. A method for treating cancer in a human patient in need thereof comprising administering an effective amount of an anti-TIGIT antibody formulation of claim 1.

30.-32. (canceled)
